(19) United States
(12) Patent Application Publication
(10) Pub. No.: US 2020/0359994 A1
(43) Pub. Date: Nov. 19, 2020

(54) SYSTEM AND METHOD FOR GUIDING ULTRASOUND PROBE

(71) Applicant: KONINKLIJKE PHILIPS N.V., EINDHOVEN (NL)

(72) Inventors: Aleksandra POPOVIC, BOSTON, MA (US); Sanghamithra KORUKONDA, SEATTLE, WA (US); Jean-luc Francois-Marie ROBERT, CAMBRIDGE, MA (US)

(21) Appl. No.: 16/762,647

(22) PCT Filed: Nov. 6, 2018
(86) PCT No.: PCT/EP2018/080321

§ 371 Date: May 8, 2020

Related U.S. Application Data
(60) Provisional application No. 62/585,000, filed on Nov. 13, 2017.

(51) Int. Cl. A61B 8/00; A61B 8/12; A61B 8/08; A61B 34/20

(52) U.S. Cl. CPC A61B 8/4218; A61B 8/12; A61B 8/54; A61B 2034/301; A61B 8/4254; A61B 34/20; A61B 8/483

(57) ABSTRACT

A controller and method for imaging an area of interest of a region within an object using a transoesophageal echo (TEE) probe of a TEE ultrasound acquisition system are 5 provided. The controller includes a memory that stores instructions, and a processor that executes the instructions. When executed by the processor, the instructions cause the controller to perform a process including causing a transthoracic echo (TTE) probe of a TTE ultrasound acquisition system to emit an ultrasound beam to a selected area of interest of a region within the object; switching the TEE probe to a listening mode, enabling the 10 TEE probe detect and receive the ultrasound beam emitted by the TTE probe; and causing a robot to steer the TEE probe to an imaging location in the object using the detected TTE ultrasound beam. The TEE probe shows the area of interest using ultrasound images acquired from the imaging location.

### TECHNICAL FIELD

This disclosure relates to minimally invasive medical procedures performed using image guidance instruments, and more particularly to systems and methods for automated steering and maintaining of endoscopic narrow field-of-view ultrasound imaging using broad field-of-view external ultrasound imaging.

### BACKGROUND

Surgical robots and/or steerable devices may be used in minimally invasive medical procedures, or interventional procedures, to improve a surgeon's dexterity inside an object (e.g., the patient or the patient's body) at a surgical site. Examples of surgical robots include multi-arm systems, such as da Vinci® robots, or flexible robots, such as Medrobotics Flex® robotic systems. These robotic systems are controlled by the user (e.g., surgeon) using different interface mechanisms, including hand controllers or input handles for the operating robotic systems. Imaging systems are also incorporated to enable visualization of areas of interest inside the object, the robotic systems and the tools controlled by the robotic systems when inside the areas of interest. Imaging systems may include ultrasound, X-rays, computed tomography (CT) scans and magnetic resonance imaging (MRI), for example.

Among the types of ultrasound systems used for interventional procedures are three-dimensional (3D) transoesophageal echo (TEE) ultrasound acquisition systems and transthoracic echo (TTE) ultrasound acquisition systems. (For simplicity, 3D TEE will be referred to simply as “TEE” throughout.) In procedures involving a patient's heart, such as in structural heart repair, for example, a TEE ultrasound acquisition system may be used to provide high resolution images of areas of interest (e.g., valves). That is, a TEE probe of the TEE ultrasound acquisition system is inserted through the patient's esophagus to provide ultrasound imaging from within the body. Due to size limitations of the TEE probe (e.g., restricted by esophagus) and close proximity to heart when inserted, the field-of-view of the TEE probe is narrow, and otherwise insufficient to image the entire heart. However, the TEE probe is able to provide high resolution images of the portions of the heart within its narrow field-of-view due in part to the close proximity of the TEE probe. The TTE ultrasound acquisition system is an external ultrasound imaging device with a broad field-of-view, commonly used to image a larger area or region of the patient, such as the entire heart and some surrounding area, e.g., in diagnostic and interventional cases. However, due in part to greater distance to heart (or other region), images provided by the TTE ultrasound acquisition system are low resolution images, which do not have sufficient detail and/or clarity of the areas of interest within the region for performing certain procedures, particularly as compared to the images provided by the TEE probe from inside the patient's body.

Generally, there are a number of different views from the TEE probe of areas of interest (targets) needed in structural heart disease interventions, including for example “en face” view of the mitral valve, mid-esophageal four-chamber view, long-axis view, transgastric view, tri-leaflet aortic valve view, x-plane view, and the like. These various views provided by the TEE probe at different locations are quite helpful to a cardiologist (or other interventionalist) for performing specific tasks within the patient, but are challenging to obtain, e.g., by an echocardiographer. Also, once the TEE probe is positioned to obtain a particular view, the TEE probe head requires continuous manual manipulation and adjustment within the patient by the echocardiographer to maintain the view. There is usually much discussion between the cardiologist and echocardiographer when determining a particular view for a specific task, and in locating and maintaining that view, since the cardiologist often provides feedback to the echocardiographer during the interventional procedure.

For improved overall ultrasound imaging, TEE and TTE images may be combined to provide narrow field-of-view high-resolution information with broad field-of-view low-resolution information. Examples of such combined ultrasound imaging are provided by international patent applications PCT/IB2014/066462 to Korukonda et al., filed Dec. 1, 2014, and PCT/IB2017/058173, filed Apr. 6, 2017, the entire contents of both of which are hereby incorporated by reference. However, in order to obtain the highest quality images, the TEE probe, in particular, needs to be optimally placed and/or repositioned in a imaging position adjacent the area of interest throughout the procedure, which may be difficult for the operator due to the narrow field-of-view.

Accordingly, it is desirable to provide systems, methods and computer-readable storage media for coordinated control of both TEE and TTE ultrasound acquisition systems to provide through automation accurate positioning of the TEE probe for high resolution imaging, despite the narrow field-of-view of the TEE probe.

### SUMMARY

According to another illustrative embodiment, a controller is provided for imaging an area of interest of a region within an object using a transoesophageal echo (TEE) probe of a TEE ultrasound acquisition system, the TEE probe being inserted in the object. The controller includes a memory that stores instructions; and a processor that executes the instructions. When executed by the processor, the instructions cause the controller to perform a process including causing a transthoracic echo (TTE) probe of a TTE ultrasound acquisition system to emit an ultrasound beam to a selected area of interest of a region within the object; switching the TEE probe to a listening mode, enabling the TEE probe to detect and receive the ultrasound beam emitted by the TTE probe; and causing a robot to steer the TEE probe to an imaging location in the object using the detected TTE ultrasound beam. The TEE probe shows the area of interest using ultrasound images acquired from the imaging location.

According to another illustrative embodiment, a method is provided for automated guidance of a TEE probe of a TEE ultrasound acquisition system to an imaging location adjacent an area of interest in an object during an interventional procedure. The method includes switching the TEE probe to a listening mode; causing emission of an ultrasound beam by a TTE probe of a TEE ultrasound acquisition system to the area of interest, the TEE probe, the TEE probe detecting the ultrasound beam emitted by the TTE probe in the listening mode; causing a robot to steer the TEE probe to the imaging location using the detected TTE ultrasound beam; and receiving ultrasound images from the TEE probe positioned at the imaging location showing the area of interest.

### BRIEF DESCRIPTION OF THE DRAWINGS

The present invention will be more readily understood from the detailed description of exemplary embodiments presented below considered in conjunction with the accompanying drawings, as follows.

FIG. 1 is a simplified block diagram showing a system for imaging an area of interest of a patient during an interventional procedure, according to a representative embodiment.

FIG. 2 is a simplified block diagram showing a portion of the system of FIG. 1, depicting movement of a TEE probe to an end position aligned with a TTE signal, according to a representative embodiment.

FIG. 3 is a simplified functional block diagram showing a feedback loop of a controller of the system of FIG. 1, according to a representative embodiment.

FIG. 4 is a flowchart showing a method of imaging an area of interest of a patient during an interventional procedure, according to a representative embodiment.

FIG. 5 is a flowchart showing a method of steering a TEE probe to an imaging location for showing the area of interest of the patient during the interventional procedure, according to a representative embodiment.

### DETAILED DESCRIPTION

The present invention will now be described more fully hereinafter with reference to the accompanying drawings, in which embodiments of the present invention are shown. The present invention may, however, be embodied in different forms and should not be construed as limited to the embodiments set forth herein. Rather, these embodiments are provided as teaching examples of the invention.

Generally, according to various embodiments, a TEE probe of a TEE ultrasound acquisition system is precisely steered at least in part by a robot using a focused ultrasound beam emitted by a TTE probe of a TTE ultrasound acquisition system. That is, a user may select an area of interest in a region of the patient's body (e.g., the heart or other organ) using a display of the TTE image, e.g., on a graphical user interface (GUI). Once the area of interest is selected, the TTE probe generates a focused ultrasound beam that targets the selected area of interest and a controller uses the focused ultrasound beam probe image to cause the robot to steer the TEE probe to an imaging location (indicated by the focused ultrasound beam) to show the selected area of interest in a resulting TEE image. The procedure may be based on acoustic servoing using an adaptive control loop, for example, which does not require explicit registration between the TEE probe and the TTE ultrasound acquisition system or the TTE probe, thus simplifying the workflow.

Notably, live ultrasound guidance, such as that provided by the TEE and/or TTE ultrasound acquisition systems, is used in variety of procedures, including interventional cardiology, oncology and surgery. For the sake of convenience, the embodiments herein will be described in the context of interventional cardiology of structural heart disease (SHD). Examples of such procedures include mitral clip deployments, transcatheter aortic valve replacements (TAVR), coronary artery bypass grafting and mitral valve replacement. However, it is understood that the embodiments may be applied to any interventional or surgical field procedures that include use of a TEE probe or other endoscopic imaging device, such as minimally invasive general surgery (e.g., laparoscopic ultrasound and GI ultrasound), prostate surgery (e.g., transrectal ultrasound and GI ultrasound), cholecystectomy, and natural orifice translumenal endoscopic surgery, for example, without departing from the scope of the present teachings.

It should be understood that the disclosure is provided in terms of medical instruments; however, the present teachings are much broader and are applicable to any imaging instruments and imaging modalities. In some embodiments, the present principles are employed in tracking or analyzing complex biological or mechanical systems. In particular, the present principles are applicable to internal tracking procedures of biological systems and procedures in all areas of the body such as the lungs, gastro-intestinal tract, excretory organs, blood vessels, etc. The elements depicted in the figures may be implemented in various combinations of hardware and software and provide functions which may be combined in a single element or multiple elements.

It should be further understood that the terminology used herein is for purposes of describing particular embodiments only, and is not intended to be limiting. Any defined terms are in addition to the technical and scientific meanings of the defined terms as commonly understood and accepted in the technical field of the present teachings.

As used in the specification and appended claims, the terms “a”, “an” and “the” include both singular and plural referents, unless the context clearly dictates otherwise. Thus, for example, “a device” includes one device and plural devices. The statement that two or more parts or components are “coupled” shall mean that the parts are joined or operate together either directly or indirectly, i.e., through one or more intermediate parts or components, so long as a link occurs.

Directional terms/phrases and relative terms/phrases may be used to describe the various elements' relationships to one another, as illustrated in the accompanying drawings. These terms/phrases are intended to encompass different orientations of the device and/or elements in addition to the orientation depicted in the drawings.

A “computer-readable storage medium” encompasses any tangible storage medium which may store instructions which are executable by a processor of a computing device. The computer-readable storage medium may be referred to as a non-transitory computer-readable storage medium, to distinguish from transitory media such as transitory propagating signals. The computer-readable storage medium may also be referred to as a tangible computer-readable medium.

In some embodiments, a computer-readable storage medium may also be able to store data which is able to be accessed by the processor of the computing device. Examples of computer-readable storage media include, but are not limited to: a floppy disk, a magnetic hard disk drive, a solid state hard disk, flash memory, a USB thumb drive, Random Access Memory (RAM), Read Only Memory (ROM), an optical disk, a magneto-optical disk, and the register file of the processor. Examples of optical disks include Compact Disks (CD) and Digital Versatile Disks (DVD), for example CD-ROM, CD-RW, CD-R, DVD-ROM, DVD-RW, or DVD-R disks. The term computer readable-storage medium also refers to various types of recording media capable of being accessed by the computer device via a network or communication link. For example, data may be retrieved over a modem, over the internet, or over a local area network. References to a computer-readable storage medium should be interpreted as possibly being multiple computer-readable storage mediums. Various executable components of a program or programs may be stored in different locations. The computer-readable storage medium may for instance be multiple computer-readable storage medium within the same computer system. The computer-readable storage medium may also be computer-readable storage medium distributed amongst multiple computer systems or computing devices.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

Computer storage is any non-volatile computer-readable storage medium. Examples of computer storage include, but are not limited to: a hard disk drive, a USB thumb drive, a floppy drive, a smart card, a DVD, a CD-ROM, and a solid state hard drive. In some embodiments computer storage may also be computer memory or vice versa. References to “computer storage” or “storage” should be interpreted as possibly including multiple storage devices or components. For instance, the storage may include multiple storage devices within the same computer system or computing device. The storage may also include multiple storages distributed amongst multiple computer systems or computing devices.

A “processor” as used herein encompasses an electronic component which is able to execute a program or machine executable instruction. References to the computing device comprising “a processor” should be interpreted as possibly containing more than one processor or processing core. The processor may for instance be a multi-core processor. A processor may also refer to a collection of processors within a single computer system or distributed amongst multiple computer systems. The term computing device should also be interpreted to possibly refer to a collection or network of computing devices each comprising a processor or processors. Many programs have instructions performed by multiple processors that may be within the same computing device or which may even be distributed across multiple computing devices.

A “user interface” or “user input device” as used herein is an interface which allows a user or operator to interact with a computer or computer system. A user interface may provide information or data to the operator and/or receive information or data from the operator. A user interface may enable input from an operator to be received by the computer and may provide output to the user from the computer. In other words, the user interface may allow an operator to control or manipulate a computer and the interface may allow the computer indicate the effects of the user's control or manipulation. The display of data or information on a display or a graphical user interface is an example of providing information to an operator. The receiving of data through a touch screen, keyboard, mouse, trackball, touchpad, pointing stick, graphics tablet, joystick, gamepad, webcam, headset, gear sticks, steering wheel, wired glove, wireless remote control, and accelerometer are all examples of user interface components which enable the receiving of information or data from a user.

A “hardware interface” encompasses an interface which enables the processor of a computer system to interact with and/or control an external computing device and/or apparatus. A hardware interface may allow a processor to send control signals or instructions to an external computing device and/or apparatus. A hardware interface may also enable a processor to exchange data with an external computing device and/or apparatus. Examples of a hardware interface include, but are not limited to: a universal serial bus, IEEE 1394 port, parallel port, IEEE 1284 port, serial port, RS-232 port, IEEE-488 port, Bluetooth connection, Wireless local area network connection, TCP/IP connection, Ethernet connection, control voltage interface, MIDI interface, analog input interface, and digital input interface.

A “display” or “display device” or “display unit” as used herein encompasses an output device or a user interface adapted for displaying images or data. A display may output visual, audio, and or tactile data. Examples of a display include, but are not limited to: a computer monitor, a television screen, a touch screen, tactile electronic display, Braille screen, Cathode ray tube (CRT), Storage tube, Bistable display, Electronic paper, Vector display, Flat panel display, Vacuum fluorescent display (VF), Light-emitting diode (LED) displays, Electroluminescent display (ELD), Plasma display panels (PDP), Liquid crystal display (LCD), Organic light-emitting diode displays (OLED), a projector, and Head-mounted display.

Like numbered elements in these figures are either equivalent elements or perform the same function. Elements which have been discussed previously will not necessarily be discussed in later figures if the function is equivalent.

Initially, it is noted that medical images may include 2D or 3D images such as those obtained via an ultrasonic transducer or an endoscopic camera provided on a distal end of an probe or an endoscope, respectively, or via a forward-looking camera provided at the distal end of a robot (e.g. as the end effector). Also, live images may include still or video images captured through medical imaging during the minimally invasive procedure. Other medical imaging may be incorporated during the surgical process, such as images obtained by externally applied ultrasound, X-ray and/or magnetic resonance, for example, for a broader view of the surgical site and surrounding areas.

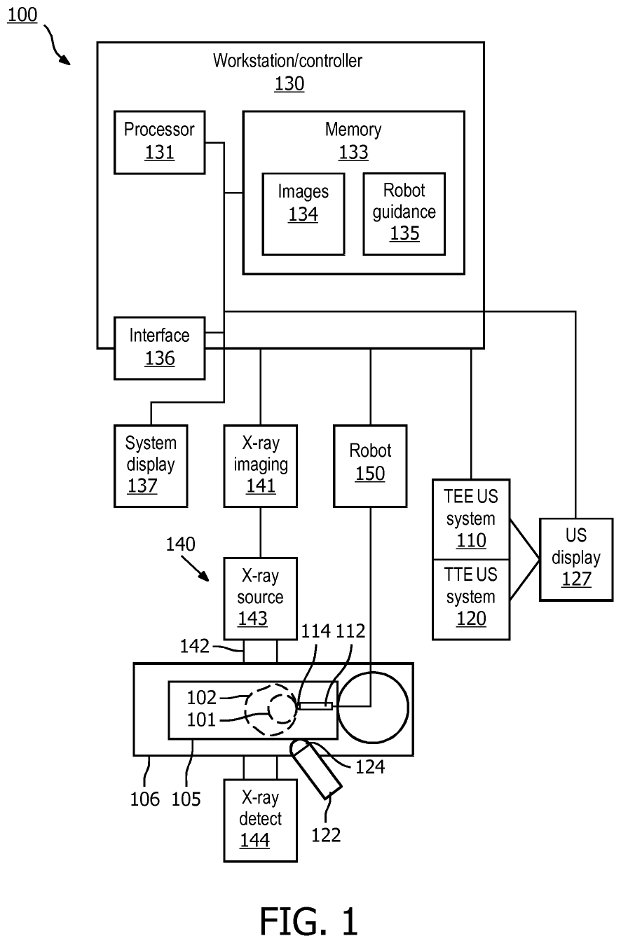

FIG. 1 is a simplified schematic diagram showing a system for imaging an area of interest of a patient during an interventional procedure, including guidance of an ultrasound imagining probe, according to a representative embodiment.

Referring to FIG. 1, a system 100 in an operating room (OR) or cathlab, for example, is used for performing an interventional procedure, such as a structural heart repair procedure, for example, on object (e.g., patient) 105 lying on table 106. Of course, the system 100 may be used for other types of interventional procedures, without departing from the scope of the present teachings. The interventional procedures typically involve manipulation of surgical instruments and other tools operable by a robot at a surgical site located within the object.

The system 100 includes TEE ultrasound acquisition system 110, TTE ultrasound acquisition system 120 and a controller (work station) 130. The controller 130 determines and controls positioning of a TEE probe 112 of the TEE ultrasound acquisition system 110, as discussed below. The controller 130 may also cause images from the TEE ultrasound acquisition system 110 and the TTE ultrasound acquisition system 120 to be displayed on an ultrasound display 127 and/or a system display 137, and to be stored in the images module 134 of memory 133. Alternatively, the TEE ultrasound acquisition system 110 and the TTE ultrasound acquisition system 120, which include the ultrasound display 127, may cause the TEE images and the TTE images to be displayed on the ultrasound display 127 directly.

The TEE ultrasound acquisition system 110 includes the TEE probe 112, which has at least one ultrasonic transducer 114 on a probe head at the distal end of the TEE probe 112. The TEE probe 112 passes through the mouth the patient (object 105) and into the esophagus to provide TEE ultrasound images an area of interest 101 (e.g., a portion of the heart) from within the object 105. The TEE ultrasound acquisition system 110 is operable to capture signals and images from the TEE probe 112, which may be displayed and/or stored, as discussed above. The TTE ultrasound acquisition system 120 includes a TTE probe 122, which has at least one ultrasound transducer 124 on a probe head at the distal end of the TTE probe 122. The TTE probe head is brought into physical contact (e.g., manually) with the exterior of the object 105 to provide TTE ultrasound images of a region 102 (e.g., the entire heart and some surrounding area), including the area of interest 101, within the object 105. The TTE ultrasound acquisition system 120 is operable to capture signals and images from the TEE probe 112, which may be displayed and/or stored, as discussed above. In an embodiment, the TEE and TTE ultrasound acquisition systems 110 and 120 may be one integrated ultrasound system.

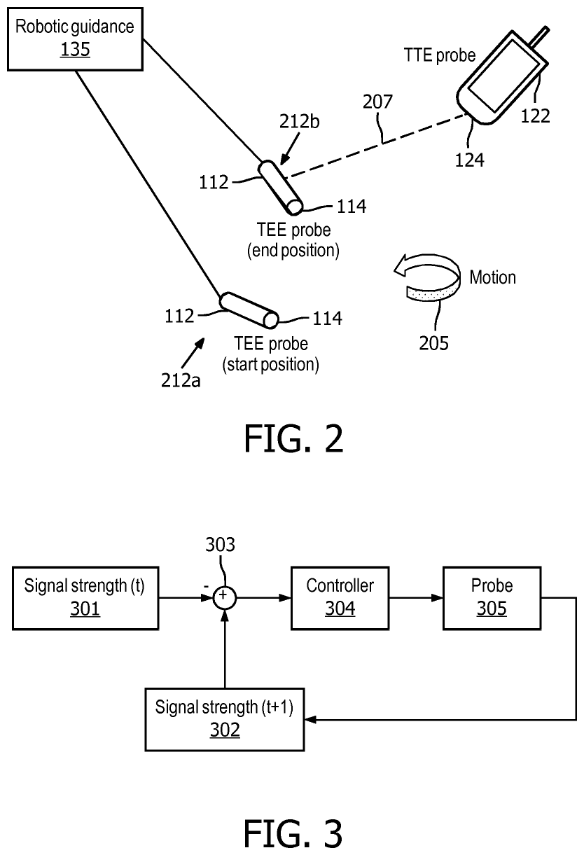

The controller 130 of the system 100 includes a processor 131, memory 133, user interface 136 and system display 137. The controller 130 may be implemented as a graphical user interface (GUI), for example. The processor 131 may be programmed to determine a target position or imaging location of the TEE probe 112 based on a selected area of interest 101 and view(s) to be shown by the TEE images (e.g., “en face” view of the mitral valve, mid-esophageal four-chamber view, long-axis view, transgastric view, tri-leaflet aortic valve view, x-plane view, and the like). For example, the processor 131 may be programmed to determine the imaging location in response to the user identifying the area of interest 101 in a TTE image from the TTE probe 122 using the user interface 136. The processor 131 is programmed to then guide the TEE probe 112, e.g., through controlling robot 150, to a focused ultrasound beam emitted by the TTE probe 122 using signal strength of the focused ultrasound beam detected by the TEE probe 112, as discussed below with reference to FIGS. 2 and 3. In an embodiment, the determination of the imaging location, the receipt and processing of information from the TEE and TTE probes 112 and 122, and the guidance of the TEE probe 112 to the imaging location are made by executing one or more computer programs stored as software on one or more computer readable storage media of the memory 133, for example.

The user interface 136 includes input device(s), such as a keyboard, a mouse, a joy stick, a haptic device, speakers, microphone or any other peripheral or control devices to permit user feedback from and interaction with the controller 130. This includes, for example, programming, providing data to, or otherwise accessing the processor 131, and managing the ultrasound and system displays 127 and 137. In various embodiments, the ultrasound display 127 may include separate displays for the ultrasound images provided by the TEE ultrasound acquisition system 110 and the TTE ultrasound acquisition system 120, respectively. Alternatively, the ultrasound display 127 (and/or the system display 137) may be a single display, in which case the controller 130 may be configured to switch between input from the TEE ultrasound acquisition system 110 and input from the TTE ultrasound acquisition system 120, respectively, such that high resolution, narrow field-of-view images, and low resolution, broad field-of-view images of the area of interest 101 may be selected, as desired. Notably, display of the TTE ultrasound image may also show the TEE probe 112 as it is guided into the area of interest 101 and positioned at the imaging location. Thus, the display of the TTE ultrasound image may be used to monitor guidance of the TEE probe 112 to one or more imaging locations for obtaining various high resolution ultrasound images throughout the procedure.

Thus, in sum, the controller 130 is configured to receive (and store) images of the region 102 from the TTE probe 122, and to enable the user to select an area of interest 101 in the received (or stored) images via the user interface 136. The controller 130 may then send a command to the TTE ultrasound acquisition system 120 to have the TTE probe 122 send a focused ultrasound beam directed only to the selected area of interest 101. The controller 130 is further configured to turn on the listening mode in the TEE probe 112 to detect the focused ultrasound beam sent by the TTE probe 122. The controller 130 repeatedly receives the listening signal from the TEE probe 112, identifies the corresponding signal strengths, and sequentially moves the TEE probe 112 (through control of the robot 150) to a position where the TEE probe 112 receives the focused ultrasound beam at maximum signal strength, through a feedback loop described for example with reference to FIGS. 2 and 3.

Additional medical equipment may be provided to enable performance of the interventional procedure by the system 100, as would be apparent to one of skill in the art. For example, as shown in FIG. 1, an x-ray scanner 140 is included for additional imaging of the area of interest 101. The x-ray scanner 140 includes an x-ray imaging controller 141 that controls operation of a c-arm 142. The c-arm 142 includes an x-ray source 143 and a corresponding x-ray detector 144, located on opposite sides of the object 105. The c-arm 142 has radiographic capabilities, and may be used for fluoroscopic imaging, for example, during surgical or diagnostic procedures, as is known to those skilled in the art. An example of a c-arm, implemented by an X-ray system, is described by Popovic, U.S. Pat. No. 9,095,252 (issued Aug. 4, 2015), which is hereby incorporated by reference. For fluoroscopic imaging, a contrast agent may be injected into the object 105. The system display 137 may display the contrast enhanced X-ray images in real time. Support systems, such as an anesthesia system (not shown) and a respiration ventilator (not shown) may be provided, as well, and operated independently or under control of the controller 130.

A typical OR staff during a structural heart repair procedure on the object 105 includes the echocardiographer, who manages the TEE and TTE probes, and the cardiologist, who navigates interventional devices, such as catheters and guidewires (not shown) from arterial incisions in the object 105 into the heart under guidance of the X-ray and/or ultrasound images in order to perform various diagnostic or therapeutic procedures. In conventional systems, the echocardiographer manually manipulates the TEE probe into a suitable position for imaging the area of interest 101, typically under the direction of the cardiologist. However, use of a TEE ultrasound acquisition system, without guidance from a TTE ultrasound acquisition system as described herein, has a number of challenges. For example, the position and orientation of the probe head of the TEE probe requires constant, minute adjustments by the echocardiographer for the duration of the interventional procedure in order to maintain appropriate visualization of the target structures. This can lead to fatigue and poor visualization during long procedures. Also, the length of the TEE probe results in the echocardiographer being positioned in close proximity to the x-ray source 143 of the c-arm 142, thus increasing exposure to x-rays over the course of the interventional procedure. Further, during certain phases of an interventional procedure, the cardiologist and echocardiographer must be in constant communication as the cardiologist instructs the echocardiographer as to which portion of the region 102 to visualize with the TEE probe. Given the difficultly interpreting a 3D ultrasound volume, and the different co-ordinate systems displayed by the x-ray and ultrasound systems, it can be challenging for the echocardiographer to understand and/or carry out the instructions of the cardiologist. In addition, when the area of interest is not visible in the TEE images due to the narrow field-of-view of the TEE probe, the echocardiographer must sweep the TEE probe in order to find the proper location for obtaining the desired images. This may be a time-consuming effort, further impacted by difficult mapping between TEE dials on the ultrasound acquisition system and the TEE probe head position.

In comparison, according to the representative embodiment shown in FIG. 1, the controller 130 determines the guidance information and controls the robot 150 to move the TEE probe 112. In particular, the robot 150 may be controlled by a robot guidance/control module 135 in the memory 133. The control information is based on changes in the signal strength of the focused ultrasound beam generated by the TTE probe 122 and detected by the TEE probe 112. The robot 150 is able to manipulate the TEE probe 112 in one or all degrees-of-freedom (e.g., two dial rotation, probe head rotation, and insertion). The controller 130 is thus operable to receive signals from TEE probe 112, and generate robot motion parameters for operating the robot 150 based on the signals from TEE probe 112.

The user (e.g., the echocardiographer and/or the cardiologist) may select the area of interest 101 in the TTE image, and the TEE probe 112 is steered to an appropriate imaging location to show the selected area of interest 101 in the TEE image provided by the TEE probe 112. Steering the TEE probe 112 is based on acoustic servoing using an adaptive control loop, as shown in FIG. 3, for example, which does not require explicit registration between the TEE probe 112 and the TTE ultrasound acquisition system 120 and/or the TTE probe 122[correct?], simplifying the workflow. More particularly, the robot guidance/control module 135 receives the signal strength information from the TEE probe 112 with respect to the focused ultrasound beam from the TTE probe 122, which targets the area of interest 101. The robot guidance/control module 135 controls the robot 150 to maneuver the TEE probe 112 through the esophagus to the imaging location and to maintain the TEE probe 112 in the imaging location throughout a session or procedure, or until a different view of the area of interest 101 or a different area of interest 101 is selected from the TEE probe 112 and/or the TTE probe 122.

FIG. 2 is a simplified block diagram of a portion of the system 100 depicting movement of the TEE probe 112 to an end position (i.e., the imaging location), aligned with a focused ultrasound beam from the TTE probe 122, according to a representative embodiment. FIG. 3 is a simplified functional block diagram showing a feedback loop of an ultrasound controller (e.g., controller 130), according to a representative embodiment.

Referring to FIG. 2, the TEE probe 112 is shown in a start position 112a and an end position 112b, moved by robot 150 as directed by robot guidance/control module 135 in a motion generally depicted by arrow 205. The end position 212b of the TEE probe 112 is identified or otherwise indicated by a focused ultrasound beam 224 generated by the at least one transducer 124 of the TTE probe 122. For example, a user (e.g., the echocardiographer and/or the cardiologist) selects the desired area of interest 101 in a TTE ultrasound image showing the region 102 (e.g., the heart), in which the selected area of interest 101 is located. The user may then manipulate the TTE probe 122 outside the object 105, e.g., manually and/or robotically, such that the focused ultrasound beam 207 emitted from the at least one transducer 124 of the TTE probe 122 points to the selected area of interest 101. Alternatively, the TTE probe 122 may be maneuvered to point to the selected area of interest 101 by fully or partially automated system, e.g., including another robot, for enhanced control of the positioning. The robot 150 then moves the TEE probe 112, which has been switched to a listening mode, in the direction of the arrow 205 until it intersects the ultrasound beam 207 at the end position 212b. The movement of the TEE probe 112 and the determination of when the TEE probe 112 has intercepted the ultrasound beam 224 results from a feedback loop 300, shown in FIG. 3, as discussed below, executed by the processor 131 in the controller 130. Once at the end position 212b, the TEE probe 112 may be switched to an active mode, in which it generates TEE ultrasound beams and receives corresponding reflected ultrasound signals to provide high resolution imaging of the area of interest 101, displayed for example on the ultrasound display 127 and/or the system display 137.

Referring to FIG. 3, the feedback loop 300 is performed by the controller 130 for controlling movement of the TEE probe 112 from the start position 212a to the end position 212b shown in FIG. 2, the end position 212b being indicated by the focused ultrasound beam 207. As stated above, the TEE probe 112 is switched to a listening mode at the start position 212a, and thus receives the focused ultrasound beam 207 (ultrasound signal) emitted by the TTE probe 122 to target the end position 212b. In an embodiment, the TEE probe 112 is initially moved, e.g., by the robot 150 or manually by the echocardiographer, along a predetermined path (not shown) until it first acquires the focused ultrasound beam 207, which marks the starting position 212a. The controller 130 interprets the focused ultrasound beam 207 received from TEE probe 112, and finds the signal peak, which is representative of signal strength of the focused ultrasound beam 207. The TEE probe 112 is then moved by operation of the robot 150 under control of the controller 130 in a direction of increasing signal strength through measurements at time increments using the feedback loop 300 until the TEE probe 112 reaches the end position 212b, at which the signal strength of the focused ultrasound beam is at its maximum.

More particularly, the signal strength of the focused ultrasound beam 207 received by the TEE probe 112 at time t is measured at block 301 (at the signal peak) by the controller 130. The signal strength of the focused ultrasound beam 207 at time t+1 is measured at block 302 by the controller 130, where the TEE probe 112 has been moved incrementally by operation of the robot 150 (e.g., along the motion arrow 205) to a new position between times t and t+1. The signal strength measured at time t+1 is summed with the signal strength measured at time t at block 303, and the resulting difference is obtained by the controller 130 at block 304. The controller 130 controls movement of the TEE probe 112 at block 305 (via the robot guidance/control module 135 and the robot 150) in response to the resulting difference in signal strength, with the goal of moving the TEE probe 112 to a next position from which the next measured signal strength will be greater than the previous measured signal strength, as determined at block 303.

For example, when the difference in signal strengths measured at time t and time t+1 is positive, indicating increased signal strength, the controller 130 causes the robot 150 to continue to move the TEE probe 112 in the same direction (or continue along a predetermined path) based on the assumption that the signal strength will continue to increase. When the difference in signal strengths measured at time t and time t+1 is negative, indicating decreased signal strength, the controller 130 causes the robot 150 to move the TEE probe 112 in the opposite direction (or back to a previous point along the predetermined path) on the assumption that the signal strength will otherwise continue to decrease. This process continues until it is determined that the maximum signal strength has been attained, in which case the controller 130 causes the robot 150 to move the TEE probe 112 the position corresponding to the maximum signal strength, or to remain in place if already in the position corresponding to the maximum signal strength.

In various embodiments, the controller 130 may cause the robot 150 to move the TEE probe 112 in some direction other than the same or opposite direction at block 305 in response to a negative difference between sequential signal strength measurements, thereby exploring various directions from a present position of the TEE probe 112 until the signal strength measurements begin to increase again or until it is determined that the present position is the position at which the TEE probe 112 receives the focused ultrasound beam 207 at maximum signal strength. Alternatively, in response to a negative difference between adjacent signal strength measurements, the controller 130 may cause the robot 150 to return the TEE probe 112 to the previous position (i.e., where it was located at time t), and then move the TEE probe 112 in some other direction from that position at the next time increment, again exploring various directions until the signal strength measurements begin to increase again or until it is determined that the present position is the position at which the TEE probe 112 receives the focused ultrasound beam 207 at maximum signal strength.

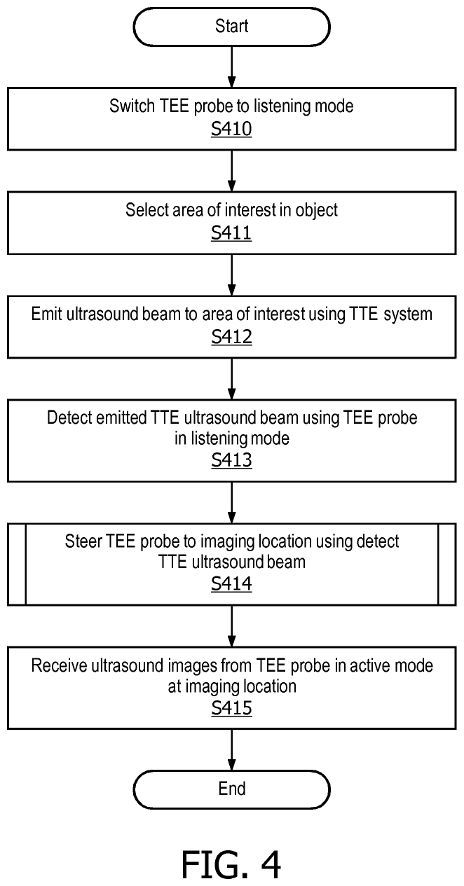

FIG. 4 is a flowchart showing a method of imaging an area of interest of a patient during an interventional procedure, according to a representative embodiment. More particularly, FIG. 4 shows positioning a TEE probe guided by a focused ultrasound beam from a TTE ultrasound system, according to a representative embodiment.

Referring to FIG. 4, a TEE probe of a TEE ultrasound acquisition system is switched to a listening mode in block S410, which may be done by the controller or by the user, either directly or through the user interface with the controller. The user selects an area of interest in a TTE ultrasound image, provided by a TTE probe of a TTE ultrasound acquisition system in block S411. The TTE probe is pressed against the object (e.g., a patient) near the internal area of interest. The TTE probe has a broad field-of-view that encompasses a region that includes multiple potential areas of interest, including the selected area of interest. The area of interest may be selected in 3D or 2D TTE ultrasound images provided by the TTE ultrasound acquisition system. In block S412, the TTE probe actively emits a focused ultrasound beam to the selected area of interest. The TTE probe generates the focused ultrasound beam using one or more ultrasound transducers along a plane of the selected area of interest.

In block S413, the focused ultrasound beam emitted by the TTE ultrasound probe is detected using the TEE probe having been switched to operate in a listening mode. In the listening mode, the TEE transducer(s) of the TEE probe are not emitting ultrasound signals, but rather are configured to receive ultrasound signals, particularly the focused ultrasound beam from TTE probe. As discussed above, the TEE probe is inserted in esophagus of the patient, and maneuvered within the esophagus by a robot under control of a controller. In an embodiment, detecting the focused ultrasound beam emitted from the TTE ultrasound probe may include moving the TEE probe (e.g., manually or under robotic control) along a predefined detection path in the patient until the focused ultrasound beam emitted from the TTE ultrasound probe is initially detected, and then stopping the TEE probe. The predefined detection path may include a set of concentric spheres around the probe head of the TEE probe, for example.

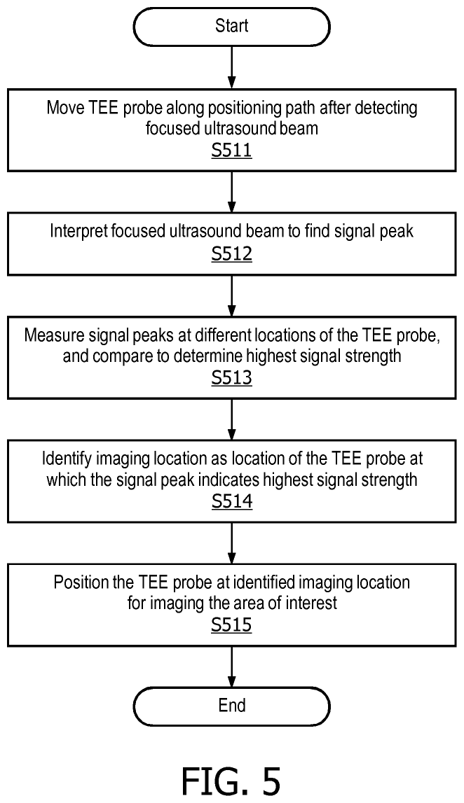

Once the focused ultrasound beam is detected, the TEE probe is steered from the position at which the focused ultrasound beam is first detected to an imaging location in block S414 using characteristics of the detected focused ultrasound beam, such as signal strength, an example of which is discussed below with reference to FIG. 5. The controller may cause the robot to steer the TEE probe to the imaging location using feedback derived from signals provided by the TEE probe, as discussed above. From the imaging location, the TEE probe is switched to an active mode, enabling it to generate ultrasound signals and to receive reflected ultrasound signals showing the area of interest at high resolution in a narrow field-of-view.

FIG. 5 is a flowchart showing a method, corresponding to block S414 of FIG. 4, of steering the TEE probe to the imaging location for showing the area of interest within the patient, according to a representative embodiment.

Referring to FIG. 5, in block S511, the TEE probe is controlled by the controller (via a robot guidance/control module and a robot) to move along a positioning path after detecting the focused ultrasound beam emitted by the TTE probe of the TTE ultrasound acquisition system, while the TEE probe continues to receive the focused ultrasound beam. The received focused ultrasound beam is interpreted in block S512 to find a signal peak, the signal peak being representative of signal strength of the received focused ultrasound beam.

In block S513, magnitudes of the signal peak are measured at different locations of the TEE probe as the TEE probe is moved along the positioning path over a series of incremental time steps, respectively, and compared to one another to determine a highest signal strength of the received focused ultrasound beam. Generally, differential of signal peaks of a current time step and the preceding time step in the control loop defines an error signal for the control loop, as discussed above with reference to FIG. 3. That is, when the direction of motion of the TEE probe is toward improving signal strength, the motion continues in the same direction. For example, for a one degree-of-freedom (DOF) robot, the direction of motion is proportional to the signal strength gradient along that direction. For a multiple DOF robot, a direction vector, the size of which is equal to the number of degrees of freedom, is proportional to the signal gradient. Once the signal gradient is negative in all directions of movement, the motion of the TEE probe is stopped as the position corresponding to the maximum signal strength has been reached. In block S514, the imaging location is identified as the location of the TEE probe at which the signal peak indicates the highest signal strength of the received focused ultrasound beam. The TEE probe is positioned at the identified imaging location in block S515 for showing the area of interest through active mode ultrasound imaging.

In an alternative embodiment, a model of the region 102 (e.g., a heart model) is overlaid on top of the TTE image provided by the TTE probe 122. The user (e.g., the echocardiographer) selects an anatomical landmark from the model of the region 102, and the anatomical landmark is transferred to a coordinate frame of the TTE image by the processor 131. The model of the region 102, including the anatomical landmarks, may be previously stored in the memory 133, and the TTE image may be stored in the memory 133 upon acquisition by the TTE probe 122. The coordinate frame of the TTE image is applied by the processor 131, and the combined coordinate frame and TTE image are likewise stored in the memory 133 to enable the processor 131 to transfer the selected anatomical landmark. In this case, the focused ultrasound beam generated by the TTE probe 122 is directed to the anatomical landmark transferred to the TTE image. The TEE probe 112 is then positioned in the imaging location (e.g., moved from the start position 212a to the end position 212b as shown in FIG. 2) using the signal strength feedback in the listening mode, as described above with reference to FIGS. 2 and 5, for example.

In practice, the discussed control processes may be implemented by modules that are embodied by any combination of hardware, software and/or firmware installed on any platform (e.g., a general computer, application specific integrated circuit (ASIC), etc.). Furthermore, processes such as those depicted in FIGS. 2-5 may be performed by the controller 130, and particularly the processor 131 in combination with the memory 133 and various interfaces. Embodiments of the disclosure may also be directed to a non-transitory computer-readable storage medium having stored therein machine readable instructions configured to be executed by the processor 131 to control the robot 150 including feedback operation of the TEE ultrasound acquisition system 110 with respect to movement of the TEE probe 112 to the imaging location indicated by the focused ultrasound beam emitted by the TTE probe 122. The corresponding machine readable instructions are configured to perform methods depicted in FIGS. 2-5, for example.

While various embodiments have been illustrated and described in detail in the drawings and foregoing description, such illustration and description are to be considered illustrative or exemplary and not restrictive; the invention is not limited to the disclosed embodiments.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A controller for imaging an area of interest of a region within an object using a transesophageal echo (TEE) probe of a TEE ultrasound acquisition system, the TEE probe being insertable in the object, the controller comprising:
a memory for storing instructions; and
a processor for executing the instructions;
wherein, when executed by the processor, the instructions cause the controller to perform a process including:
causing a transthoracic echo (TTE) probe of a TTE ultrasound acquisition system to emit an ultrasound beam to a selected area of interest of a region within the object;
switching the TEE probe to a listening mode, enabling the TEE probe to detect and receive the ultrasound beam emitted by the TTE probe; and
causing a robot to steer the TEE probe to an imaging location in the object using the detected TTE ultrasound beam, the TEE probe providing ultrasound images of the area of interest acquired from the imaging location.

2. The controller of claim 1, wherein causing the robot to steer the TEE probe comprises using instructions of a robot guidance/control module in the memory.

3. The controller of claim 1, wherein causing the robot to steer the TEE probe to the imaging location for showing the area of interest comprises the controller to perform a process including:
causing the robot to control the TEE probe to move along a positioning path after the TEE probe detects the ultrasound beam emitted by the TTE probe, while the TEE probe continues to receive the ultrasound beam;
interpreting the received ultrasound beam to find a signal peak of current representative of signal strength of the received ultrasound beam;
measuring magnitudes of the signal peak at different locations of the TEE probe on the positioning path over a series of time steps, respectively;
identifying the imaging location as a location among the different locations at which the signal peak indicates a highest signal strength of the received ultrasound beam; and
causing the robot to position the TEE probe at the imaging location for showing the area of interest.

4. The controller of claim 3, wherein identifying the imaging location comprises the instructions causing the controller to perform a process including:
determining a differential between the magnitude of the signal peak of the TEE probe measured at a current location and the magnitude of the signal peak of the TEE probe measured at a previous location during a preceding consecutive time step;
when the differential is positive, indicating improved signal strength, causing the robot to control the TEE probe to move from the current location in a same direction as a previous movement to an updated location;
when the differential is negative, causing the robot to control the TEE probe to move from the current location to the previous location, and to move from the previous location in a different direction to the updated location;
measuring the magnitude of the signal peak of the TEE probe at a next consecutive time step in the updated location;
repeating determining the differential and causing the robot to control the TEE probe to move to another updated location until the differential is negative after moving the TEE probe in all of a predetermined number of different directions from the updated location, respectively; and
identifying the updated location as the imaging location.

5. The controller of claim 1, wherein the ultrasound beam is emitted by the TTE probe along a plane of the area of interest.

6. The controller of claim 1, wherein the TEE probe is enabled to detect the ultrasound beam emitted from the TTE probe by the instructions causing the controller to perform a process including:
causing the robot to move the TEE probe along a predefined detection path until the ultrasound beam emitted from the TTE ultrasound acquisition system is detected; and
causing the robot to stop the TEE probe at a position on the predefined detection path at which the ultrasound beam is detected.

7. The controller of claim 6, wherein the predefined detection path comprises a set of concentric spheres around a probe head of the TEE probe.

8. The controller of claim 4, wherein the robot comprises a one degree-of-freedom robot, and the controller causes the robot to move the TEE probe in a direction of motion proportional to signal strength gradient along the direction of motion.

9. The controller of claim 4, wherein the robot comprises a multiple degrees-of-freedom robot, and controller causes the robot to move the TEE probe by a direction vector proportional to signal gradient.

10. A method for automated guidance of a transesophageal echo (TEE) probe of a TEE ultrasound acquisition system to an imaging location adjacent an area of interest in an object during an interventional procedure, the method comprising:
switching the TEE probe to a listening mode;
causing emission of an ultrasound beam by a transthoracic echo (TTE) probe of a TTE ultrasound acquisition system to the area of interest, the TEE probe detecting the ultrasound beam emitted by the TTE probe in the listening mode;
causing a robot to steer the TEE probe to the imaging location using the detected TTE ultrasound beam; and
receiving ultrasound images from the TEE probe positioned at the imaging location showing the area of interest.

11. The method of claim 10, wherein causing the robot to steer the TEE probe to the imaging location comprises:
controlling the robot to move the TEE probe along a positioning path after the TEE detects the ultrasound beam probe, while the TEE probe continues to receive the ultrasound beam emitted by the TTE probe;
receiving the ultrasound beam from the TEE probe, and interpreting the received ultrasound beam to find a signal peak of current representative of signal strength of the received ultrasound beam;
determining magnitudes of the signal peaks corresponding to different locations of the TEE probe on the positioning path over a series of time steps, respectively;
identifying the imaging location as a location among the different locations at which the signal peak indicates a highest signal strength of the received ultrasound beam; and
causing the robot to position the TEE probe at the imaging location for showing the area of interest.

12. The method of claim 11, wherein identifying the imaging location comprises:
determining a differential between the magnitude of the signal peak of the TEE probe measured at a current location and the magnitude of the signal peak of the TEE probe measured at a previous location during a preceding consecutive time step;
when the differential is positive, indicating improved signal strength, controlling robot to move the TEE probe from the current location in a same direction as a previous movement to an updated location;
when the differential is negative, controlling robot to move the TEE probe from the current location to the previous location, and further to move the TEE probe from the previous location in a different direction to the updated location;
measuring the magnitude of the signal peak of the TEE probe at a next consecutive time step in the updated location;
repeating determining the differential and controlling the robot to move the TEE probe to another updated location until the differential is negative after the TEE probe is moved in all of a predetermined number of different directions from the updated location, respectively; and
identifying the updated location as the imaging location.

13. The method of claim 10, wherein the ultrasound beam is emitted by the TTE probe along a plane of the area of interest.

14. The method of claim 10, wherein the ultrasound beam emitted from the TTE probe is detected by the TEE probe by causing the robot to move the TEE probe along a predefined detection path until the TEE probe first detects the ultrasound beam, and then causing the robot to stop the TEE probe at a corresponding position on the predefined detection path.

15. The method of claim 14, wherein the predefined detection path comprises a set of concentric spheres around a probe head of the TEE probe.

16. The method of claim 10, wherein the area of interest is initially selected using a three-dimensional and/or a two-dimensional image of the object provided by the TTE ultrasound acquisition system.

17. The method of claim 10, wherein the area of interest is initially selected using a three-dimensional and/or a two-dimensional image of the object provided by the TTE probe.

18. The method of claim 12, wherein the robot comprises a one degree-of-freedom robot, and wherein a direction of motion of the TEE probe is proportional to signal strength gradient along the direction of motion.

19. The method of claim 12, wherein the robot comprises a multiple degrees-of-freedom robot, and wherein a direction vector of moving the TEE probe is proportional to signal gradient.

20. A system for imaging an area of interest of an object during an interventional procedure, the system comprising:
a transthoracic echo (TTE) ultrasound acquisition system comprising a TTE probe having at least one transducer element for emitting a focused ultrasound beam to an area of interest in an object;
a transesophageal echo (TEE) ultrasound acquisition system comprising a TEE probe insertable in the object and configured to detect the ultrasound beam emitted from the TTE probe, wherein the TEE probe is steerable to an imaging location adjacent the area of interest using the detected TTE ultrasound beam for determining the imaging location, and to provide ultrasound imaging of the area of interest from the imaging location; and
a controller comprising a processor, a robot guidance/control module and a display, the controller configured to:
cause the TTE probe to emit an ultrasound beam along a plane of the area of interest;
cause the robot guidance/control module to control a robot to move the TEE probe along a predetermined detection path in a listening mode to detect the emitted ultrasound beam;
cause the robot guidance/control module to control the robot to incrementally move the TEE probe to different locations, after detection of the emitted ultrasound beam;
determine the imaging location based on comparisons of signal strengths of the emitted ultrasound beam received by the TEE probe at the different locations, respectively, the imaging location being a location among the different locations at which the signal strength of the emitted ultrasound beam is determined to be highest;
cause the robot guidance/control module to control the robot to move the TEE probe to the imaging location for the TEE probe to obtain ultrasound images of the area of interest; and
display the ultrasound images of the area of interest obtained from the TEE probe located at the imaging location.
